(19) United States
(12) Patent Application Publication
(10) Pub. No.: US 2005/0182028 A1
(43) Pub. Date: Aug. 18, 2005

(54) Pharmaceutical formulation for oral delivery of bisphosphates

(76) Inventor: Chih-Ming James Chen, Irvine, CA (US)

Correspondence Address: Daniel B. Schein, Ph.D., Esq., P.O. Box 28403, San Jose, CA, 95159, US

(21) Appl. No.: 10/778,521

(22) Filed: Feb. 13, 2004

(51) Int. Cl. A61K 31/785; A61K 31/675; A61K 31/663

(52) U.S. Cl. 514/89; 514/102; 424/78.27

(57) ABSTRACT

The present invention discloses a method for treating or preventing a bone disorder in a mammal in need thereof comprising orally administering to said mammal a pharmaceutically effective amount of a pharmaceutical composition of at least one bisphosphonate, or a pharmaceutically acceptable salt or esters thereof, and at least one aminoalky methacrylate copolymer, according to a dosing schedule having a dosing interval selected from once-weekly dosing, twice-monthly dosing, once-monthly, once-quarterly and once-annually dosing. The present invention further discloses a method for treating or preventing a bone disorder in a mammal in need thereof comprising continuously orally administering a unit dosage per-day to said mammal in a short time for a long time therapy.

### REFERENCE CITED

United States Patent Documents

20020150624 A1
U.S. Pat. No. 6,372,728 B1
   Other Publications
Crandall C. Risedronate: a clinical review. Arch Intern Med. 2001; 161:353-360.
Berenson J R. Advances in the biology and treatment of myeloma bone disease. Am. J. Health Syst Pharm. 2001; 58(suppl 3):S16-S20.
Lin, J H, Bisphosphonates: A Review of their pharmacokinetic properties. Bone 18:75-85, 1996.
Merck & Co., Inc. Package literature for Fosamax®. January 2001.
Procter & Gamble Pharmaceuticals. Package literature for Acetonel. March 2001.
Janner M, Muhlbauer R C, Fleisch H. Sodium EDTA enhances intestinal absorption of two bisphosphonates, Calcif Tissur Int 1991; 49:280-283.
Gertz B J, Holland S D, Kline W F, Matuszewski B K, Freeman A, Quan H, et al. Studies of the oral bioavailability of alendronate. Clin Pharmacol Ther 1995; 58:288-298.

### BACKGROUND OF THE INVENTION

1. Field of Invention

The present invention relates to an regular dose oral formulations of bisphosphonate and their methods of use to treat/prevent diseases related to bone remodeling or bone disorders, such as for example, Paget's disease, osteoporosis, metastatic bone disease, and the like, while minimizing the potential for esophageal irritation and other adverse gastrointestinal effects. These methods comprise orally administering to a mammal in need thereof a pharmaceutically effective amount of the pharmaceutical composition of at least one bisphosphonate, or a pharmaceutically acceptable salt thereof and at least one aminoalky methacrylate copolymer, as a unit dosage according to a continuous schedule having a once-weekly, twice-weekly, biweekly, twice-monthly, once-monthly, once-quarterly or once-annually dosing interval. The present invention also relates to pharmaceutical compositions of the bisphosphonate for carrying out these methods.

2. Description of Related Art

Bisphosphonates are a group of synthetic bone-seeking compounds, which are used to treat and prevent various disorders of bones, including osteoporosis, metastatic bone Disease, and Paget's disease of bone. The pharmacological properties of bisphosphonates depend on their molecular structure consisting of the essential bisphosphonate moiety and various substitutions at R₁ and R₂, shown in a general form in Formula 1.

The use of bisphosphonates in the treatment of diseases characterized by abnormal bone remodeling is documented in H. Fleisch, “Bisphosphonates: Mechanisms of Action,” Exp. Opin. Ther. Patents, 11: 1371-1381 (2001) and H. Fleisch, “Bisphosphonates in Bone Disease,” 4^{th} edition, Academic Press, New York, 2000. Despite their proven therapeutic benefits in patients with disorders associated with abnormal bone remodeling, bisphosphonates are poorly absorbed from the gastrointestinal tract, which is further compromised by the presence of food and beverages other than water which can bind the bisphosphonates and further diminish their bioavailability. To facilitate adequate absorption from the gastrointestinal tract, it is generally recommended that oral bisphosphonates, such as alendronate and risedronate, be taken in the fasting state, 30 minutes before the first food, beverage, or medication of the day. However, many patients find the need for such fasting on a daily basis to be inconvenient. Moreover, oral administration has been associated with adverse gastrointestinal effects, especially those relating to the esophagus. The adverse gastrointestinal effects always easily cause esophageal irritation. This limitation on the use of oral bisphosphonates is a source of considerable inconvenience to patients in need of therapy and can result in decreased compliance. Furthermore, esophageal irritation has been observed with oral bisphosphonate use, albeit infrequently [for alendronate use, see P. C. De Groen, “Esophagitis associated with the use of alendronate,” New Engl. J. Med., 335: 1016-1021 (1996)].

Intravenous administration has been used to overcome this bioavailability problem. However, intravenous administration is costly and inconvenient, especially when the patient must be given an intravenous infusion lasting several hours on repeated occasions.

| | |
|---|---|
| | 1 |
| 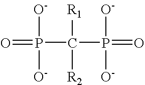 | |

Formula 1. General Structure and Representative Members of
Bisphosphonates.

| | Bisphosphonate | R₁ | R₂ |
|---|---|---|---|
| First Generation | Etidronate | OH | CH3 |
| | Clodronate | Cl | Cl |
| Second | Pamidronate | OH | CH₂CH₂CH₃ |
| Generation | Alendronate | OH | CH₂CH₂CH₂NH₂ |
| | Tiludronate | H | 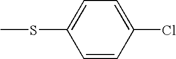 |
| Third Generation | Risedronate | OH | 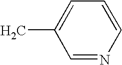 |

It is believed that the substitution at R₁ is responsible for uptaking and binding to bone mineral as well as inhibition of growth of calcium crystals; while the substitution at R₂ prevents bone resorption. The oral bioavailabilityies of bisphosonates are extremely poor lying between 1% and 10% or even lowre. For instance, alendronate and risedronate are the widely used, orally administrated agents for prevention and treatment of osteoporosis. Their oral bioavailabilities are merely 0.5%-1% and 0.63%. The absorption would be further deducted if the bisphosphonates were given with meals, milk, coffee, orange juice, calcium, or other multivalent inorganic cations (e.g. Magnesium), due to the formation of insoluble complex.

Regimens wherein the bisphosphonate, such as alendronate and risedronate, is administered at a relatively high unit dose but at a low relative dosing frequency cause less adverse gastrointestinal effects, particularly esophageal effects, compared to the administration of a low relative dose at a high relative dosing frequency. Consequently, there is a need for pharmaceutical formulations, preferably for oral administration, which is capable of enhancing the bioavailability of bisphosphonates, such as alendronate, so that the patients are able to take the medication in a more convenient manner, e.g. with a less quantity or in a less frequency. Only a few methods have been studied for enhancing the oral bioavailability, including co-administration of EDTA (which chelates calcium, consequently increases the absorption of biophosphonate), increasing in the intraluminal pH (such as H₂ receptor antagonist therapy), and using medium chain glycerides as absorption enhancer. Shunsuke Watanabe et al. also disclosed a pharmaceutical composition containing aminoalkyl methacrylate copolymer E for oral use with improved absorption by increasing drug permeability of the digestive tract mucous membrane and/or the mucous layer distributed over this mucous membrane in U.S. 2002/0150624 A1.

### SUMMARY OF THE INVENTION

The present invention relates to an regular dose oral formulations of bisphosphonate and their methods of use to treat/prevent diseases related to bone remodeling or bone disorders, such as for example, Paget's disease, osteoporosis, metastatic bone disease, and the like, while minimizing the potential for esophageal irritation and other adverse gastrointestinal effects. These methods comprise orally administering to a mammal in need thereof a pharmaceutically effective amount of the pharmaceutical composition of at least one bisphosphonate, or a pharmaceutically acceptable salt thereof and at least one aminoalky methacrylate copolymer, as a unit dosage according to a continuous schedule having a once-weekly, twice-weekly, biweekly, twice-monthly, once-monthly, once-quarterly or once-annually dosing interval. The present invention also relates to pharmaceutical compositions of the bisphosphonate for carrying out these methods. The bisphosphonate is selected from the group consisting of alendronate, cimadronate, clodronate, tiludronate, etidronate, ibandronate, risedronate, piridronate, pamidronate, zoledronate, pharmaceutically acceptable salts or esters thereof, and mixtures thereof.

Due to the solubility, most bisphosphonates are used as the forms of their salts, such as alendronate sodium and risedronate sodium. Without trying it down to any specific theory, it is believed that bisphosphonates as the anionic substances could potentially be carried by a cation-bearing compound or a mixture of cation-bearing compounds resulting in a conjugate with a better in vivo absorption. Aminoalkylmethacrylate copolymers and their derivatives are easily protonated on the amine group to form the cationic functional group, which may complex with the anionic functional groups of bisphosphonate through the ion-pair. It is found in the present invention that the oral absorption of alendronate was enhanced only by the aminoalkylmethacrylate copolymer (Eudragit E PO) but not enhances by the ammonioalkyl methacrylate copolymer dispersion (Eudragit RL 30D).

The pharmaceutical formulation according to the invention is adapted for oral administration and may be given during faster or fed conditions. In the preparation of pharmaceutical formulations according to the invention, the dosage form may exist as liquid, solid, and semi-solid. The bisphosphonate to absorption enhancing agent ratio should be between 1:10 to 10:1, preferably 1:2 to 1:5. In the form of liquid, the pH of solution should be within a range of 3-7 in order to avoid local irritation of the upper gastrointestinal mucosa. The pharmaceutical formulation, according to the invention, should be administrated orally accompanied with at least 100 mL water.

The bisphosphonate and absorption enhancing agent may be formulated to form a complex in vivo, preferably in vitro. The invention further comprises other pharmaceutically acceptable ingredients, such as filler, surfactant, disintegration agent, flavor, etc.

One embodiment of the invention relates to a method for treating or preventing a bone disorder in a mammal in need thereof comprising orally administering to said mammal a pharmaceutically effective amount of a liquid pharmaceutical composition of at least one bisphosphonate, or a pharmaceutically acceptable salt thereof, as a unit dosage which ranges from greater than about 17.5 mg to about 2000 mg, on a bisphosphonic acid active basis and absorption enhancing agent, aminoalkylmethacrylate copolymer, according to a dosing schedule having a dosing interval selected from once-weekly dosing, twice-weekly dosing, biweekly dosing, twice-monthly dosing, once-monthly dosing, once-quarterly dosing and once-annually dosing. Especially, a unit dosage which ranges from greater than about 70 mg to about 140 mg is adapted for a dosing schedule having a dosing interval once-monthly. A unit dosage, which ranges from greater than about 210 mg to about 420 mg is adapted for a dosing schedule having a dosing interval once-quarterly, and a unit dosage which ranges from greater than about 840 mg to about 1680 mg is adapted for a dosing schedule having a dosing interval once-annually. However, the less frequent dosing is possible because of long half-life (>10 years) of alendronate. This is also supported by the concept for two strengths of marketed products, Fosamax (Merck), 10 mg and 70 mg. The 10 mg strength is given daily while 70 mg strength is given weekly.

Furthermore, a continuous treatment regimens use of a once-monthly dosage in a short time, such as 2-12 days or more days, provided for a long-time therapy, such as 2 months, half year, one year and so on, is possible. This continuous treatment regimen is possible because of long half-life (>10 years) of alendronate, too.

These and other features, aspects, and advantages of the present invention will become better understood with reference to the following description and appended claims.

It is to be understood that both the foregoing general description and the following detailed description are by examples, and are intended to provide further explanations of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 depicts the cumulative amount of alendronic acid excreted in urine over a period of 8 hours;

FIG. 2 illuminate the comparison of the histomorphometries of the bone of the mouse belonging to the Group Sham, Group OVX, Group A-7 and Group C-7. Compared with sham-operated rats (Sham), ovariectomy (OVX) caused a significant loss of trabecular bone;

FIGS. 3A-B illuminate comparison of the effects of solution A (Alendronate absorption improvement formulation) and solution C (alendronate control) on the bone volume and bone mineral content (BMC) in ovariectomized rats; and

FIGS. 4A-B illuminates comparison of the effects of solution A (Alendronate absorption improvement formulation) and solution C (alendronate control) on the biomechanical properties in ovariectomized rats.

### DESCRIPTION OF THE PREFERRED EMBODIMENTS

Several different formulations are provided as following:

Formulation A: A suspension prepared with 1 alendronate sodium tablet (Fosamax®) containing alendronate sodium equivalent to 70 mg alendronie acid, 1 gram ammonioalkyl methacrylate copolymer dispersion (Eudragit RL 30D), and 50 mL of distilled water.

Formulation B: A suspension prepare with one alendronate sodium tablet (Fosamax®) containing alendronate sodium equivalent to 70 mg alendronic acid, 7 mL of 0.1N HCl solution, 210 mg of aminoalkyl methacrylate copolymer (Eudragit E PO), and 43 mL of distilled water.

Formulation C: A suspension prepared with one alendronate sodium tablet (Fosamax®) containing alendronate sodium equivalent to 70 mg alendronic acid, and 50 mL distilled water.

### EXAMPLE 1

Preparation of Suspension A

TABLE 1
Compositions of Suspension A

| Item | Ingredient | Quantity Per Dose |
|---|---|---|
| | Alendronate sodium tablet | 1 tablet containing |
| | (Fosamax ®) | alendronate sodium |
| | | equivalent to 70 mg |
| | | alendronic acid. |
| | Ammotionlkyla methacrylate | 1 g (approximately 0.3 gm |
| | copolymer dispersion (Eudragit | ammonicallky1 methacrylate |
| | RL 30D) | copolymer in solid) |
| | Distilled Water | 50 mL |

Manufacturing process includes transferring 1.0 g of ammonioalkyl methacrylate copolymer dispersion (Eudragit RL 30D) to a 100 mL glass vial. Add 50.0 mL of distilled water using a graduate cylinder to the vial and handshake the vial to obtain homogeneous polymer dispersion. Place one alendronate sodium tablet containing alendronate sodium equivalent to 70 mg alendronic acid into the vial and handshake the vial until the tablet is completely disintegrated prior to dosing.

Preparation of Formulation B

TABLE 2
Compositions

| Item | Ingredient | Quantity Per Dose |
|---|---|---|
| 1 | Alendronate Sodium tablet (Fosamax ®) | 1 tablet containing |
| | | aleadronate sodium |
| | | equivalent to 70 mg |
| | | alendronic acid |
| 2 | Aminoalky methacrylate copolymer | 210 mg |
| | (Eudragit E PO) | |
| 3 | 0.1N HC1 Solution | 7 mL |
| 4 | Distilled Water | 43 mL |

Manufacturing process includes transferring 210 mg of aminoalkyl methacrylate copolymer (Eudragit E PO) to a 100 mL glass vial. Add 7 mL of 0.1N HC1 solution to dissolve aminoalkyl methacrylate copolymer (Eudragit E PO) in the vial. After the solid is completely dissolved, pour 43 mL of distilled water using a graduate cylinder into the vial and handshake the vial to obtain homogeneous polymer solution. Place one alendronate sodium tablet containing alendronate sodium equivalent to 70 mg alendronic acid in the vial and handshake the vial until the tablet is completely disintegrated prior to dosing.

Preparation of Formulation C

TABLE 3
Compositions

| Item | Ingredient | Quantity Per Dose |
|---|---|---|
| 1 | Alendronate Sodium tablet (Fosamax ®) | 1 tablet containing |
| | | alendronate sodium |
| | | equivalent to 70 mg |
| | | glendratric acid |
| 2 | Distilled Water | 50 mL |

Place on alendronate sodium tablet containing alendronate sodium equivalent to 70 mg alendronic acid into a 100 mL glass vial to which 50 mL of distilled water is added. Handshake the vial until the tablet is completely disintegrated prior to dosing.

Bioavailability Study

An open label and randomized three-way crossover study was employed to investigate the bioavailability of Formulations A, B, and C, seven (7) healthy, adult, male subjects randomly received the three separate formulations in assigned periods, one per period. The formulations were separated by a washout period of at least seven (7) days. Drug administration consisted of an oral single suspension dose taken on the Study Day of Periods I, II, and III. Each study period consisted of one (1) drug dose, and was then accompanied by 150 mL (5 fl. ozs) of room temperature tap water, following an overnight fast. Urine samples were obtained at Hour 0 (pre-dose) and following the dose at Hours 1, 2, 3, 4, 6, and 8 and were subsequently assayed for alendronic acid.

Tables 4, 5, and 6 summarize the quantity of alendronic acid excreted in urine for Formulation A, B, and C, respectively.

TABLE 4
SUMMARY OF ALENDRONIC ACTD QUANTITY
EXCRETED IN HUMAN URINE FOR FORMULATION A

| | Sample Collection (Interval) | | | | | | |
|---|---|---|---|---|---|---|---|
| Subject # | 0 | 1.00 | 2.00 | 3.00 | 4.00 | 6.00 | 8.00 |
| 1 | BLQ | 15.19 | 42.62 | 34.54 | 14.71 | 15.24 | 6.08 |
| 2 | BLQ | 15.05 | 56.06 | 37.94 | 18.45 | 22.01 | 10.30 |
| 3 | BLQ | 15.89 | 67.73 | 42.03 | 24.17 | 12.39 | 10.19 |
| 4 | BLQ | K | 30.21 | 10.13 | 5.10 | K | 6.10 |
| 5 | BLQ | 9.06 | 22.90 | 8.98 | 7.05 | 3.44 | 3.08 |
| 6 | BLQ | 0.00 | 39.04 | 13.16 | 7.50 | 0.00 | 8.14 |
| 7 | 0.40 | 14.95 | 29.85 | 25.48 | 13.55 | 10.94 | 4.68 |
| Meau | 0.006 | 14.03 | 41.20 | 24.61 | 12.93 | 12.80 | 6.94 |

BLQ: Below Limit of Quantitation
K: Sample not collected

TABLE 5
SUMMARY OF ALENDRONIC ACTD QUANTITY
EXCRETED IN HUMAN URINE FOR FORMULATION B

| | Sample Collection (Interval) | | | | | | |
|---|---|---|---|---|---|---|---|
| Subject # | 0 | 1.00 | 2.00 | 3.00 | 4.00 | 6.00 | 8.00 |
| 1 | BLQ | 32.85 | 90.14 | 41.13 | 17.16 | 14.52 | 8.60 |
| 2 | BLQ | 82.07 | 91.52 | 29.95 | 25.94 | 28.87 | 12.19 |
| 3 | BLQ | 138.25 | 93.95 | 54.16 | 25.65 | 34.31 | 16.86 |
| 4 | BLQ | K | 244.98 | 51.21 | 33.44 | 22.15 | 10.14 |
| 5 | BLQ | 151.07 | 173.71 | 84.65 | 47.20 | 49.12 | 29.67 |
| 6 | BLQ | 97.79 | 167.46 | 88.66 | 39.58 | 24.94 | 15.67 |
| 7 | BLQ | 83.39 | 75.63 | 60.64 | 25.77 | 34.95 | 13.67 |
| Mean | 0 | 97.57 | 133.91 | 58.63 | 31.11 | 29.84 | 15.26 |

BLQ: Below Limit of Quantitation
K: Sample not collected

TABLE 6
SUMMARY OF ALENDRONIC ACTD QUANTITY
EXCRETED IN HUMAN URINE FOR FORMULATION C

| | Sample Collection (Interval) | | | | | | |
|---|---|---|---|---|---|---|---|
| Subject # | 0 | 1.00 | 2.00 | 3.00 | 4.00 | 6.00 | 8.00 |
| 1 | BLQ | 15.26 | 31.33 | 28.95 | 16.55 | 13.92 | 3.91 |
| 2 | BLQ | 14.86 | 38.05 | 22.10 | 9.72 | 8.26 | 5.68 |
| 3 | BLQ | 18.33 | 22.98 | 11.78 | 10.06 | 5.75 | 5.82 |
| 4 | BLQ | K | 52.64 | 14.91 | K | 2.62 | 17.57 |
| 5 | BLQ | 7.73 | 24.73 | 5.31 | 9.74 | 4.06 | 2.41 |
| 6 | BLQ | 23.46 | 0.00 | 32.29 | 5.53 | 3.93 | 1.20 |
| 7 | 0.55 | 5.19 | 30.79 | 36.08 | 18.71 | 22.97 | 8.89 |
| Meau | 0.09 | 12.11 | 28.65 | 21.63 | 10.04 | 8.93 | 6.50 |

BLQ: Below Limit of Quantitation
K: Sample not collected

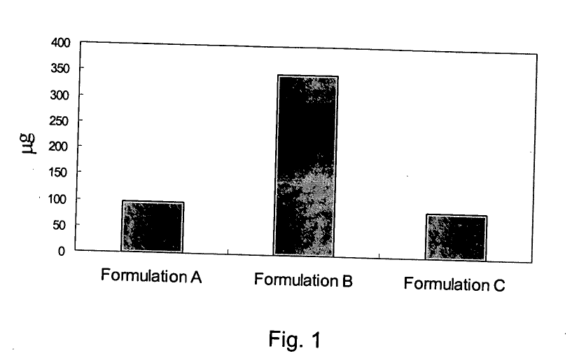

FIG. 1 depicts the cumulative amount of alendronic acid excreted in urine over a period of 8 hours. Of three formulations, Formulation B produced the highest systemic uptake of alendronate as was evidenced the amount of alendronic acid excreted in urine. The peak amount of alendronic acid in urine was detected at Hour 2 for all three formulations. Total amount of alendronic acid excreted in 8 hours after the administration of Formulation B was approximately 3-4 times greater than for Formulation A and C. The higher renal excretion excretion rate and amount strongly reveals that aminoalkyl methacrylate copolymer as one of cation-bearing compounds significantly improve the absorption of bisphosphonate.

The relative bioavailabilities of the three formulations are calculated based on its cumulative total amount of alendronate acid excreted in the urine. The statistical summary is listed in table 7.

TABLE 7

| Formulations | Geomatric Means | Ratios (%) |
|---|---|---|
| A | 96.4 | 110.9 |
| B | 346.4 | 398.3 |
| C(control) | 87.0 | □ |

*Relative bioavailability of Formulation A or B TO formulation C (control)

As shown in table 7, the cumulated urine excreted alendronate is significantly higher for Formulation B than that of Formulations A and C. The amount of alendronate excreted in urine is a reflection of its oral bioavailability The almost four-fold increase in bioavailability for Formulation B indicates its dose of 70 mg is almost equivalent to given four tablets of Fosamax® 70 mg. Furthermore, because of its extremely long terminal half-life of alendronate, i.e., more than ten years, Fosamax® 70 mg is administered weekly. This is an improved product from the original product, Fosamax® 10 mg which is administered daily. Both strengths of Fosamax® 10 and 70 mg are on the U.S. market. Assuming the linearity of oral absorption and its efficacy applicable to Formulation B, and because of its extremely long half-life, the super-bioavailability of Formulation B will provide a novel product that can be administered less frequently, e.g., monthly (because same dose of 70 mg in Formulation B will last four times longer due to its four-fold increase in bioavailability) or annually (if given higher dose, e.g., at 910 mg alendronate, i.e., (70/4)×52).

Another invention of this patent is it provides a convenient novel oral administration frequency, i.e., monthly or annually, as oral dosage form. The less frequent dosing of this unique Formulation B will provide convenience to the patients and increase their compliance, and therefore reduce the health cost and improve the quality of life for patients in the long run.

### EXAMPLE 2

Surgery and Treatment

Female Sprague-Dawley rats (3-month-old) weighing 300˜320 gm were used for this study. Rats were ovariectomized (OVX) bilaterally under trichloroacetaldehyde (200 mg/kg) anesthesia and control rats were sham-operated (Sham) for comparison. The animals were all kept under controlled conditions at room temperature (22±1° C.) and a 12-hr light-dark cycle. Animals were fed with Purina Laboratory Rodent Diet (PMI; St. Louis, Mo.) (0.95% calcium) and water ad libitum. Body weight of the rats was determined weekly. Rats were randomLy divided into 5 groups as follows.

| | | Treatment (p.o., fasting | |
|---|---|---|---|
| | | 4 hrs before and 2 hrs | |
| | | after drug treatment) | Dose-interval |
| Group | Surgery | (1 mg/Kg) | (day) |
| Sham-operated | SHAM | Vehicle | 2 |
| OVX | OVX | Vehicle | 2 |
| C-2 | OVX | Alendronate control | 2 |
| C-7 | OVX | Alendronate control | 7 |
| A-7 | OVX | Alendronate absorp. improve | 7 |

Test Solution

Solution A is prepared for the mice of Group A-7. Manufacturing process includes transferring 210 mg of aminoalkyl methacrylate copolymer (Eudragit E PO) to a 100 mL glass vial. Add about 1.2 mL of 0.1N HC1 solution to dissolve aminoalkyl methacrylate copolymer (Eudragit E PO) in the vial. After the solid is completely dissolved, pour 35 mL of distilled water using a graduate cylinder into the vial and handshake the vial to obtain homogeneous polymer solution. 91.37 mg alendronate sodium trihydrate, which contains alendronate sodium equivalent to 70 mg alendronic acid, is placed in the vial and shakes the vial until the alendronate sodium trihydrate is completely disintegrated prior to dosing. Vanilla 105 mg and sucralose 17.4 mg are added in the solution.

Solution C is prepared for the mice of Groups C-2 and C-7. Manufacturing process includes transferring 91.37 mg alendronate sodium trihydrate, which contains alendronate sodium equivalent to 70 mg alendronic acid, is placed in the vial and shakes the vial until the alendronate sodium trihydrate is completely disintegrated prior to dosing. Vanilla 105 mg and sucralose 17.4 mg are added in the solution.

Analysis of Bone Mineral Density (BMD) and Content (BMC)

At the end of the program (6 weeks after ovariectomy), the rats were sacrificed by decapitation. The tibia and femur were removed and cleaned of soft tissue, the length and weight of the tibia and femur were measured with a precision caliper (±0.05 mm) as described by Weinreb et al. (1991). B MD and BMC of the tibia were measured with a dual-energy X-ray absorptiometer (DEXA, XR-26; Norland, Fort Atkinson, Wis.). The mode adapted to the measurements of small subjects was adopted. A coefficient of variation of 0.7% was calculated from daily measurements of BMD on a lumbar phantom for more than 1 year (Yang et al., 1998). The whole tibia and femur were scanned and BMD and BMC were measured by absorptiometer.

Histomorphometry of Bone

Tibiae were fixed by 4% formaldehyde and then decalcified with 0.5N hydrochloric acid and dehydrated in an ascending series of ethanol solution and acetone, and embedded in paraffin. Serial sections (5 mm) were cut longitudinally and stained with Mayer's hematoxylin-eosin solution. Images of the growth plate and proximal tibia were photographed using Olympus microscope. Bone volume was measured in the secondary spongiosa, which is located under the primary spongiosa and characterized by a network of larger trabeculae. Bone volume was measured using image analysis software (Image-pro plus 3.0).

Biomechanical Three-Point Bending Test

Mechanical properties of bone tissues were measured in three-point bending in a material testing system (MTS-858, MTS System Inc., Minneapolis, Minn.). The span of the two support points is 20 millimeters and the deformation rate is 1 mm/min. Load/deformation curves are transported to a computer and acquired by Team 490 software (version 4.10, Nicolet Instrument Technologies Inc., Madison, Wis.). Cross-sectional parameters were measured from the photographs and used in the calculation of the cross-sectional moment of inertia. The cross-sectional moment of inertia was calculated under the assumption that the cross-sections were elliptically shaped (Turner et al., 1992):
I=π[(ab3.(a.2t)(b.2t)3)/64
where a is the width of the cross section in the mediolateral direction, b is the width of the cross section in the anteroposterior direction, and t is the average of the cortical thickness. All of these parameters are obtained using the image software Image Pro Plus 3.0 for Windows (Media Cybernetics, Silver Spring, Md.). The maximal stress, ultimate stress, and elastic modulus (Young's modulus) were calculated using the following equations (Turner and Burr, 1993):
   σ=FLc/4I
   E=F/dL3/48I
where σ is ultimate stress, c is the distance from the center of mass (equal to ½b as described above), F is the applied load (N), d is the displacement (mm), and L is the span between the two support points of the bending fixture (mm). In addition, the energies to ultimate stress are also measured by computing the respective areas under the stress-strain curve.
   Results
   Prevention of Bone Loss by Alendronate Control and Alendronate Absorption Improvement Formulation

The results are organized in the table 8, table 8 illuminates the treatment/prevention of bone loss by Alendronate absorption improving formulation.

TABLE 8
Prevention of bone loss by Alendronate absorption improving
formulation in ovariectomized (OVX) rats

| Group | Sham | OVX | C-2 | C-7 | A-7 |
|---|---|---|---|---|---|
| Bone length, mm | | | | | |
| Tibia | 4.06 | 4.06 | 4.05 | 3.96 | 4.12 |
| Femur | 3.68 | 3.64 | 3.66 | 3.65 | 3.76 |
| Wet weight, mg | | | | | |
| Tibia | 762.6 | 667.1 | 758.6 | 677.5 | 770.7 |
| Femur | 998.9 | 883 | 958.3 | 883.6 | 990.0 |
| BMD, g/cm3 | | | | | |
| Tibia | 0.112 | 0.104 | 0.115 | 0.109 | 0.108 |
| Femur | 0.139 | 0.129 | 0.137 | 0.133 | 0.137 |
| BMC, g | | | | | |
| Tibia | 0.330 | 0.275 | 0.315 | 0.277 | 0.302 |
| Femur | 0.443 | 0.381 | 0.424 | 0.399 | 0.424 |
| Bone volume, % | 19.1 | 8.6 | 20.9 | 15.7 | 19.1 |
| Young's modulus, | 18.8 | 10.1 | 20.8 | 9.4 | 18.2 |
| GPa | | | | | |
| Ultimate stress, | 187.6 | 109.1 | 208.4 | 93.9 | 182.1 |
| MPa | | | | | |

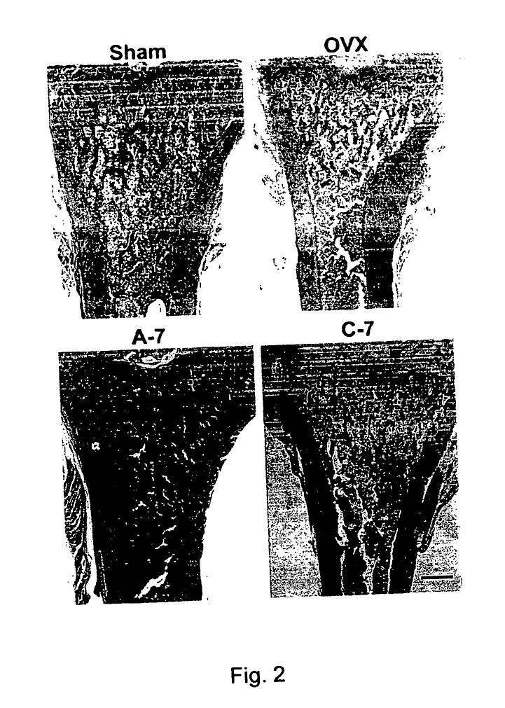

Rats showed decrease of wet weight in both femur and tibia after 6 weeks' ovariectomy. Treatment with alendronate control on alternate days (C-2) or Alendronate absorption improving formulation once per week (A-7) prevented the weight loss of both tibia and femur in OVX rats. FIG. 2 illuminated the comparison of the histomorphometries of the bone of the mouse belonging to the Group Sham, Group OVX, Group A-7 and Group C-7. Compared with sham-operated rats (Sham), ovariectomy (OVX) caused a significant loss of trabecular bone. In comparison with alendronate control (C-7), once/week treatment of solution A (Alendronate absorption improvement formulation), Group A-7 is more efficient in the prevention of the loss of trabecular bone in secondary spongiosa. Bar in the picture of C-7 equals 0.5 mm.

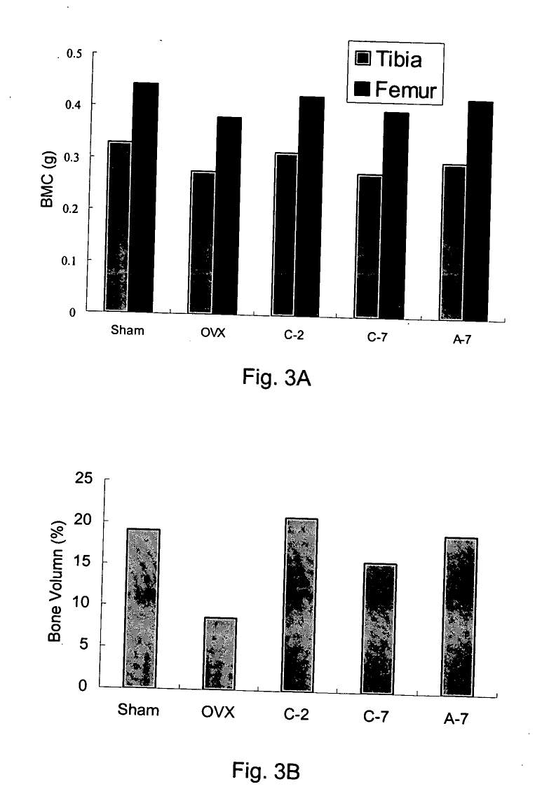

Referring is made to FIGS. 3A-B, FIGS. 3A-B illuminates comparison of the effects of solution A (alendronate absorption improvement formulation) and solution C (alendronate control) on the bone volume and bone mineral content (BMC) in ovariectomized rats. Ovariectomy for 6 weeks reduced BMC, illuminated in FIG. 3A, and bone volume, illuminated in FIG. 3B in both tibia and femur. Alendronate control treated on alternate days (C-2) is much more efficient to prevent bone loss induced by ovariectomy than that treatment on once/week (C-7). On the other hand, alendronate absorption improvement formulation treated on once/week (A-7) can markedly inhibit the OVX-induced decrease of BMC and bone volume. The bone volume decreases by 55% in response to OVX. Treatment with alendronate control on alternate days (C-2) completely reversed the loss of bone volume. However, when the dose interval was increased from 2 to 7 days (C-7) the protection effect was significantly decreased. However, once/week treatment with solution A. (alendronate absorption improvement formulation) (Group A-7) completely antagonized the loss of bone volume. These results suggest that solution A is more effective than solution C (alendronate control) to prevent the bone loss induced by QVX when treated at longer dosing intervals. We further examined the effect of alendronate on the bone mineral content (BMC). It was shown in FIG. 3A and table 8 that OVX decreased BMD and BMC in both tibia and femur. Treatment with alendronate control on alternate days but not on once/week efficiently prevents the loss of bone mineral content. On the other hand, once/week treatment with solution A (alendronate absorption improvement formulation) markedly antagonized the decrease of BMD and BMC caused by ovariectomy.

Effect of Solution a (Alendronate Absorption Improvement Formulation) on the Biomechanical Properties in OVX Rats

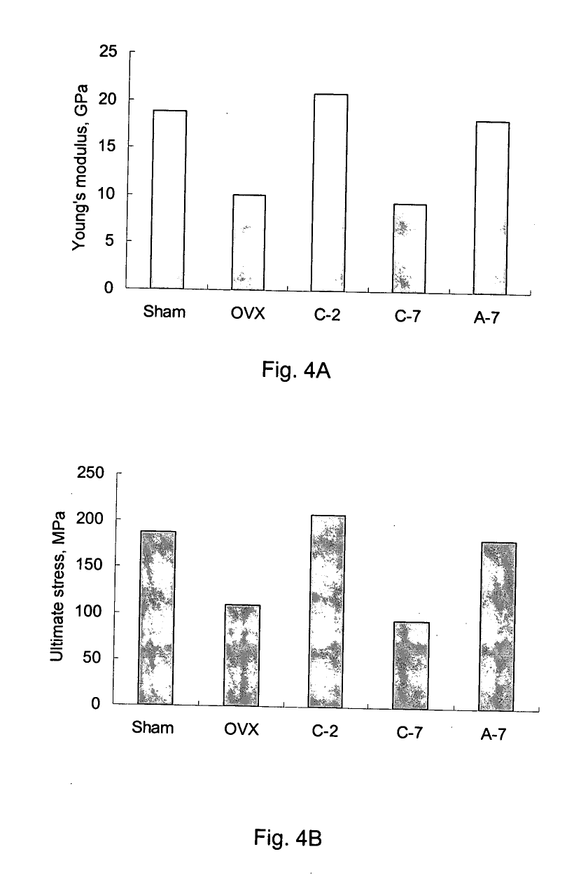

Referring is made to FIGS. 4A-B, FIGS. 4A-B illuminates comparison of the effects of solution A (Alendronate absorption improvement formulation) and solution C (alendronate control) on the biomechanical properties in ovariectomized rats. Ovariectomy for 6 weeks decreased Young's modulus, illuminated in FIG. 4A, and ultimate stress, illuminated in FIG. 4B in femur upon three-point bending test. Alendronate control treated on alternate days (C-2) is much more efficient to prevent the decrease of bone strength induced by ovariectomy than that treatment on once/week (C-7). On the other hand, Alendronate absorption improvement formulation treated on once/week (A-7) can efficiently inhibit the OVX-induced decrease of Young's modulus and ultimate stress.

Three-point bending test was done in the femur. Compared with Sham-operated group, the ultimate stress and Young's modulus decreased in OVX rats. Treatment with alendronate on alternate days but not on once/week exhibited protection against OVX-induced decrease of bone strength. On the other hand, once/week treatment with solution A (Alendronate absorption improvement formulation) completely reversed the effect of OVX on the biomechanical properties.

### DISCUSSION AND CONCLUSION

OVX rats have been shown to reproducibly lose bone mass from the axial and appendicular skeleton with declining levels of systemic estrogen similar to postmenopausal women (Kimmel, 1996). The OVX rat thus has become a useful animal model that mimics the bone loss observed for postmenopausal women. Skeletal pharmacology studies in OVX rats have been predictive of clinical efficacy in postmenopausal women for bisphosphonates (Seedor et al., 1991). Comparing the metabolism speed between mouse and human being, the mouse is at least 4 times faster than human being. The dose in the solution C is once-weekly for human being. Therefore, the dose in the solution A is adapted for a dosing schedule having a dosing interval once-monthly.

Here we found that OVX reduced BMD, BMC, bone volume and biomechanical properties of the long bones. Alendronate control treated on alternate days but not once/week (1 mg/kg) treatment efficiently prevents the bone loss. However, solution A (Alendronate absorption improvement formulation), whose oral absorption has been enhanced for several folds, can significantly prevent ovariectomy-induced bone loss even if administered once per week. Therefore, solution A (Alendronate absorption improvement formulation) may be used clinically at longer dosing intervals than alendronate control.

Another invention of this patent is it provides a convenient novel oral administration frequency, i.e., quarterly or annually, as oral dosage form. The less frequent dosing of this unique solution A will provide convenience to the patients and increase their compliance, and therefore reduce the health cost and improve the quality of life for patients in the long run. The once-annually dosage provide by increasing the dose 12 folds (e.g., given larger volume of oral solution at higher drug concentration, it is expected to have similar efficacy because of its long half-life.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A method for treating or preventing a bone disorder in a mammal in need thereof comprising orally administering to said mammal a pharmaceutically effective amount of a pharmaceutical composition of at least one bisphosphonate, or a pharmaceutically acceptable salt or esters thereof, and at least one aminoalky methacrylate copolymer, according to a dosing schedule having a dosing interval selected from once-weekly dosing, twice-monthly dosing, once-monthly, once-quarterly and once-annually at low doses.

2. The method according to claim 1 wherein said bisphosphonate is selected from the group consisting of alendronate, cimadronate, clodronate, tiludronate, etidronate, ibandronate, risedronate, piridronate, pamidronate, zoledronate, pharmaceutically acceptable salts or esters thereof, and mixtures thereof.

3. The method according to claim 2 wherein said pharmaceutically acceptable salt is alendronate monosodium trihydrate.

4. The method according to claim 2, wherein the once-monthly dosing of alendronate range is about 70 mg to about 140 mg.

5. The method according to claim 2, wherein the once-quarterly dosing of alendronate range is about 210 mg to about 420 mg.

6. The method according to claim 2, wherein the once-annually dosing of alendronate range is about 840 mg to about 1680 mg.

7. The method according to claim 1, wherein the bisphosphonate to the aminoalky methacrylate copolymer ratio should be between 1:10 to 10:1.

8. The method according to claim 1, wherein the bisphosphonate to the aminoalky methacrylate copolymer ratio should be preferably between 1:2 to 1:5.

9. The method according to claim 1, wherein the pharmaceutical composition can be manufactured as a liquid, semisolid or solid dosage forms.

10. A method for treating or preventing a bone disorder in a mammal in need thereof comprising orally administering to said mammal once-monthly a unit dosage of a pharmaceutical composition of alendronate, or a pharmaceutically acceptable salt or esters thereof, in which contains equivalent to 70 mg to 140 mg alendronic acid, and at least one aminoalky methacrylate copolymer, in which the alendronic acid to the aminoalky methacrylate copolymer ratio should be preferably between 1:2 to 1:5.

11. A method for treating or preventing a bone disorder in a mammal in need thereof comprising orally administering to said mammal once-quarterly a unit dosage of a pharmaceutical composition of alendronate, or a pharmaceutically acceptable salt or esters thereof, in which contains equivalent to 210 mg to 420 mg alendronic acid, and at least one aminoalky methacrylate copolymer, in which the alendronic acid to the aminoalky methacrylate copolymer ratio should be preferably between 1:2 to 1:5.

12. A method for treating or preventing a bone disorder in a mammal in need thereof comprising orally administering to said mammal once-annually a unit dosage of a pharmaceutical composition of alendronate, or a pharmaceutically acceptable salt or esters thereof, in which contains equivalent to 840 mg to 1680 mg alendronic acid, and at least one aminoalky methacrylate copolymer, in which the alendronic acid to the aminoalky methacrylate copolymer ratio should be preferably between 1:2 to 1:5.

13. A method for treating or preventing a bone disorder in a mammal in need thereof comprising continuously orally administering a unit dosage per-day to said mammal in a short time for a long time therapy, the unit dosage of a pharmaceutical composition of alendronate, or a pharmaceutically acceptable salt or esters thereof, in which contains equivalent to 70 mg to 140 mg alendronic acid, and at least one aminoalky methacrylate copolymer, in which the alendronic acid to the aminoalky methacrylate copolymer ratio should be preferably between 1:2 to 1:5.

14. The method according to claim 13, wherein the short time is between 2 days and 12 days, and the corresponsive long time therapy is between 2 months to one year.
